(19) United States
(12) Patent Application Publication
(10) Pub. No.: US 2016/0303357 A1
(43) Pub. Date: Oct. 20, 2016

(54) BIOLOGICAL SAMPLING PLATFORM AND PROCESSES FOR MAKING AND USING SAME

(71) Applicants: SUBHADIP BODHAK, GAITHERSBURG, MD (US); NATIONAL INSTITUTE OF STANDARDS AND TECHNOLOGY, Gaithersburg, MD (US)

(72) Inventors: Carl G. Simon, JR., Olney, MD (US); Subhadip Bodhak, Gaithersburg, MD (US); Luia A. Fernandez de Castro Diaz, Washington, DC (US); Pamela Gehron Robey, Bethesda, MD (US)

(21) Appl. No.: 14/686,269

(22) Filed: Apr. 14, 2015

(51) Int. Cl. A61M 31/00; A61B 5/00

(52) U.S. Cl. CPC A61M 31/005; A61B 5/0071; A61B 5/4848; A61B 2090/3937

(57) ABSTRACT

A biological sampling platform includes a substrate and a first through hole disposed in the substrate to receive a first sample and to provide the first sample to a biological system in response to the biological sampling platform being disposed in the biological system, the substrate being cleavable to provide a discrete layer having a thickness effective for analysis of the discreet layer by transmission microscopy. A process for collecting a biological sample includes disposing a first through hole in a substrate; disposing a first sample in the first through hole to form a biological sampling platform; disposing the biological sampling platform in a biological system; providing the first sample to the biological system in response to the biological sampling platform being disposed in the biological system; and receiving a first biological sample from the biological system in the first through hole to collect the first biological sample.

### STATEMENT REGARDING FEDERALLY SPONSORED RESEARCH

This invention was made with United States government support from the National Institute of Standards and Technology. The government has certain rights in the invention.

### BRIEF DESCRIPTION

Disclosed is a biological sampling platform comprising: a substrate; and a first through hole disposed in the substrate to receive a first sample and to provide the first sample to a biological system in response to the biological sampling platform being disposed in the biological system, the substrate being cleavable to provide a discrete layer having a thickness effective for analysis of the discreet layer by transmission microscopy.

Further disclosed is a biological sampling platform comprising: a substrate; a first through hole disposed in the substrate; and a first sample disposed in the first through hole, the first sample being provided to a biological system in response to the biological sampling platform being disposed in the biological system.

Also disclosed is a process for making a biological sampling platform, the process comprising: disposing a first through hole in a substrate; and disposing a first sample in the first through hole, the substrate being cleavable to provide a discrete layer having a thickness effective for analysis of the discreet layer by transmission microscopy

Additionally disclosed is a process for collecting a biological sample, the process comprising: disposing a first through hole in a substrate; disposing a first sample in the first through hole to form a biological sampling platform; disposing the biological sampling platform in a biological system; providing the first sample to the biological system in response to the biological sampling platform being disposed in the biological system; and receiving a first biological sample from the biological system in the first through hole to collect the first biological sample, the substrate being cleavable to provide a discrete layer having a thickness effective for analysis of the discreet layer by transmission microscopy.

### BRIEF DESCRIPTION OF THE DRAWINGS

The following descriptions should not be considered limiting in any way. With reference to the accompanying drawings, like elements are numbered alike:

FIGS. 1A and 1B respectively show a top view (FIG. 1A) and a cross-section (FIG. 1B) of an embodiment of a biological sampling platform;

FIG. 2A shows a top view of an embodiment of a biological sampling platform;

FIGS. 2B and 2C show cross-sections of alternative embodiments of the biological sampling platform shown in FIG. 2A;

FIGS. 3A and 3B respectively show a top view (FIG. 3A) and a cross-section (FIG. 3B) of an embodiment of a biological sampling platform;

FIG. 4 shows a top view of an embodiment of a biological sampling platform;

FIGS. 5A and 5B show embodiments of a biological sampling platform;

FIG. 6 shows an embodiment of a biological sampling platform;

FIG. 7A shows a perspective view of a biological sampling platform;

FIG. 7B shows a cross-section along plane X-Z that bisects the biological sampling platform shown in FIG. 7A;

FIG. 7C shows a cross-section along plane Y-Z that bisects the biological sampling platform shown in FIG. 7A;

FIGS. 8A and 8B respectively show embodiments of a biological sampling platform;

FIG. 9 shows an X-ray image of a biological sampling platform disposed in an organism according to Example 2;

FIGS. 10A and 10B respectively show a perspective and top view of an X-ray computed 3D tomography of bone formed in a subcutaneous space of a mouse according to Example 3;

FIG. 11 shows a photograph of a slide a discrete layer from a biological sampling platform disposed on a slide for optical microscopy according to Example 3; and

FIG. 12 shows a photograph of a discrete layer from a biological sampling platform according to Example 4.

### DETAILED DESCRIPTION

A detailed description of one or more embodiments is presented herein by way of exemplification and not limitation.

It has been discovered that a biological sampling platform described herein provides high-throughput screening, e.g., in vivo screening, of a physiological response, e.g., osteogenesis. According to an embodiment, the biological sampling platform includes a material selected for compatibility with biological tissue, e.g., not eliciting a substantial negative immunological or physiological response. The biological sampling platform can be implanted, e.g., subcutaneously into an organism to provide high-throughput screening or to decreases contribution of organism-to-organism variability. The biological sampling platform can be machined to produce discreet layer for histological analysis that is fast and systematic. In a particular embodiment, the biological sampling platform is used in high-throughput in vivo screening of osteogenesis, in vivo potency test for stem cells, scaffold formulations, and the like.

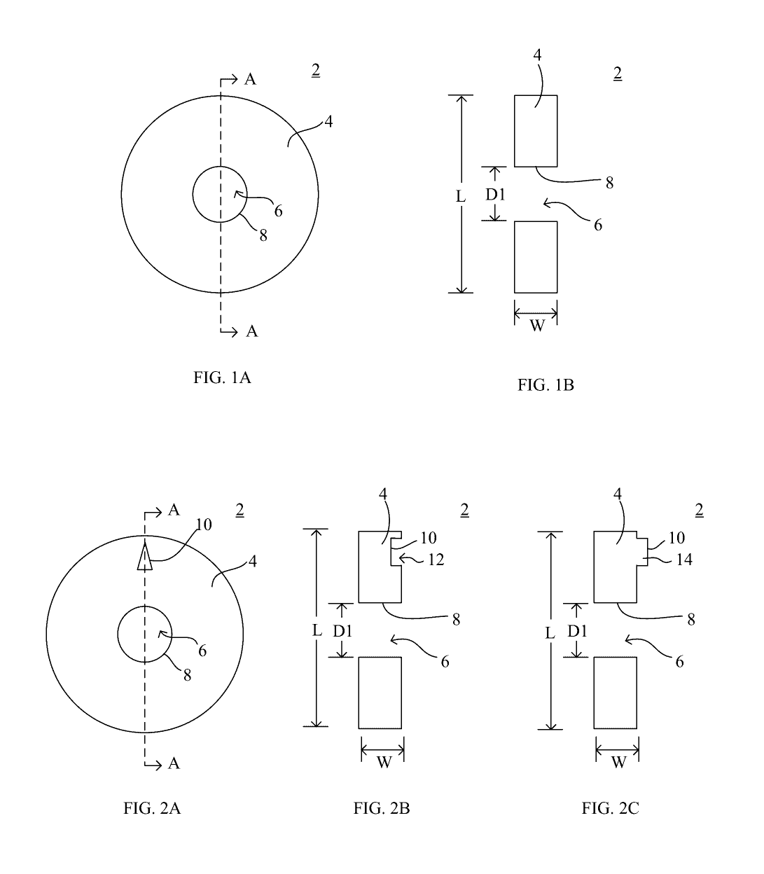

In an embodiment, as shown in FIG. 1A (top view of a biological sampling platform 2) and FIG. 1B (cross-section of biological sampling platform 2 along line A-A shown in FIG. 1A), biological sampling platform 2 includes substrate 4 and first through hole 6 disposed in substrate 4. First through hole 6 is bounded by wall 8 such that first through hole 6 includes first diameter D1. Substrate 4 has thickness W and size L, corresponding to a largest linear dimension of substrate 4. First through hole 6 is configured to receive a first sample and to provide the first sample to a biological system in response to biological sampling platform 2 being disposed in the biological system. Moreover, substrate 4 is cleavable to provide a discrete layer having a thickness effective for analysis of the discreet layer by transmission microscopy. A position of first through hole 6 can be selected such that first through hole 6 is symmetrically or asymmetrically disposed in substrate 4.

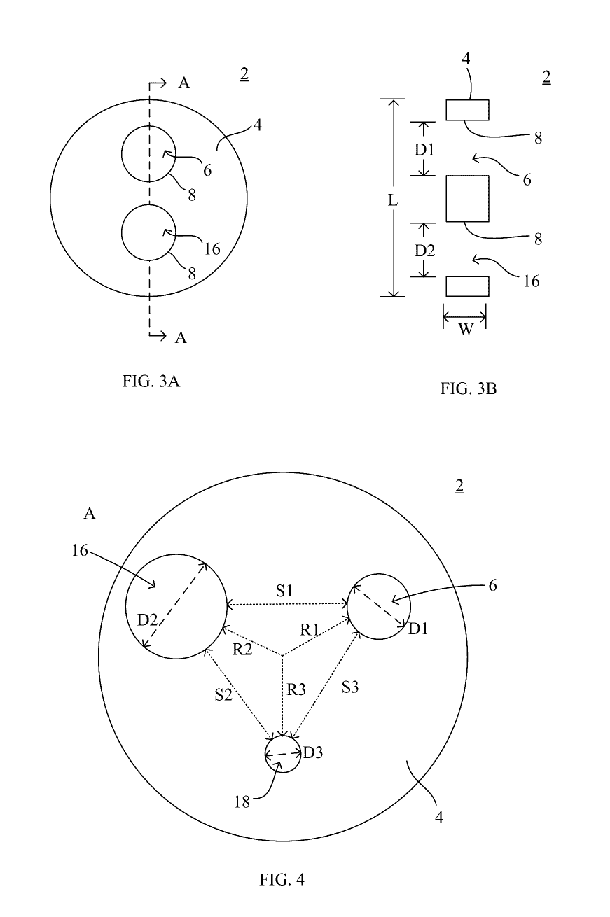

According to an embodiment shown in FIG. 2A (top view of a biological sampling platform 2), biological sampling platform 2 includes fiduciary mark 10 disposed on substrate 4. A position (e.g., proximity to an edge of substrate 4), shape (e.g., circular, polygonal, and the like), or size of fiduciary mark 10 can be selected so that fiduciary mark 10 is useful to index an orientation of biological sampling platform 2, e.g., in an environment such as in vivo or in vitro. It is contemplated that fiduciary mark 10 is useful to identify individual through holes in an embodiment of biological sampling platform 2 having a plurality of through holes (see, e.g., an embodiment of biological sampling platform 2 shown in FIG. 3A). Fiduciary mark 10 can include inset 12 or protrusion 14 as shown respectively in FIG. 2B (which is a cross-section along line A-A of an embodiment of biological sampling platform 2) and FIG. 2C (which is a cross-section along line A-A of an alternative embodiment biological sampling platform 2).

In an embodiment, as shown in FIG. 3A (top view of a biological sampling platform 2) and FIG. 3B (cross-section of biological sampling platform 2 along line A-A shown in FIG. 3A), biological sampling platform 2 includes second through hole 16 disposed in substrate 4. Second through hole 16 is configured to receive a second sample and to provide the second sample to the biological system in response to biological sampling platform 2 being disposed in the biological system. Here, second through hole 16 is bounded by wall 8 such that second through hole 16 includes second diameter D2. It should be appreciated that a shape and size of first through hole 6 and second through hole 16 independently can be any shape and size effective to respectively receive the first sample and the second sample. First diameter D1 and second diameter D2 are shown as being a dimensional size of first through hole 6 and second through hole 16 and can be a diameter (e.g., when a shape of first through hole 6 or second through hole 16 have a round shape) or a largest linear dimension.

In some embodiments, biological sampling platform 2 includes a plurality of through holes, e.g., first through hole 6, second through hole 16, and third through hole 18 having third diameter D3. Through holes (6, 16, 18) can be spaced apart from one another by a selected distance, e.g., first distance S1, second distance S2, or third distance S3, which can be a same distance or different distance. Additionally, through holes (6, 16, 18) can be arranged in an arbitrary pattern in substrate 4. In a particular embodiment, through holes (6, 16, 18) can be displaced from a location, e.g., central location, of substrate 4 respectively by a selected amount such as first radius R1, second radius R2, or third radius R3, which can be a same displacement or different displacement.

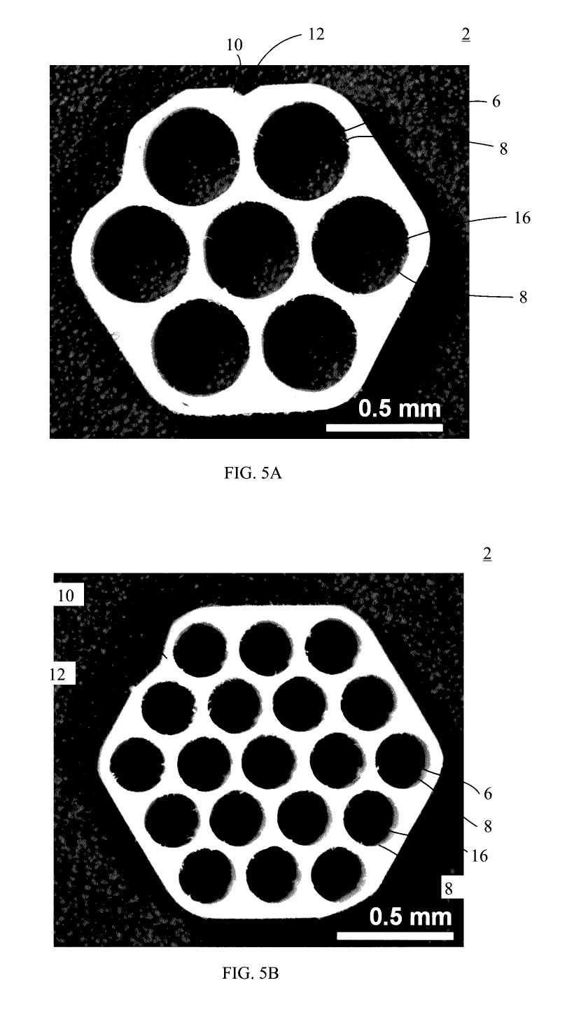

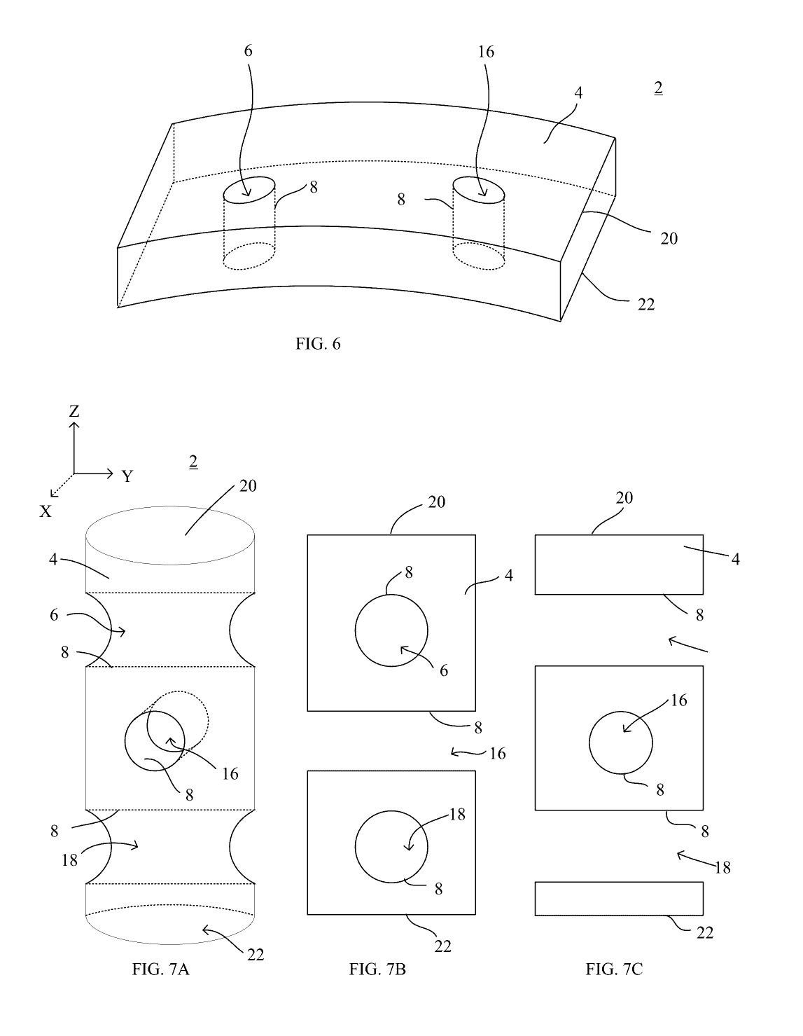

Biological sampling platform 2 can be provided in various formats. Exemplary formats include a planar substrate 4 (e.g., biological sampling platforms 2 shown in FIGS. 5A-5D) or curved substrate 4 (e.g., biological sampling platforms 2 shown in FIG. 6 or FIG. 7A) having a selected number of through holes. According to an embodiment, biological sampling platform 2 includes a plurality of through holes such as 7 through holes (FIG. 5A, photograph of biological sampling platform 2) or 19 through holes (FIG. 5B, photograph of biological sampling platform 2), and the like without limitation.

According to an embodiment, as shown in FIG. 6 (which is a perspective view of biological sampling platform 2), substrate 4 is curved and has first surface 20 and second surface 22. First through hole 6 is disposed in substrate 4 and traverses thickness W from first surface 20 to second surface 22. A number of through holes disposed in substrate 4 is not limited, e.g., biological sampling platform 2 also can include second through hole 16.

With reference to FIG. 7A (a perspective view of biological sampling platform 2), in a certain embodiment, biological sampling platform 2 includes substrate 4 that has a cylindrical shape with through holes (6, 16, 18) disposed along a length of substrate 4. Through holes (6, 16, 18) can be arranged such that a length axis of through holes (6, 16, 18) are parallel or at a selected angle. FIG. 7B shows a cross-section along plane X-Z that bisects biological sampling platform 2 shown in FIG. 7A. Similarly, FIG. 7C shows a cross-section along plane Y-Z that bisects biological sampling platform 2 shown in FIG. 7A.

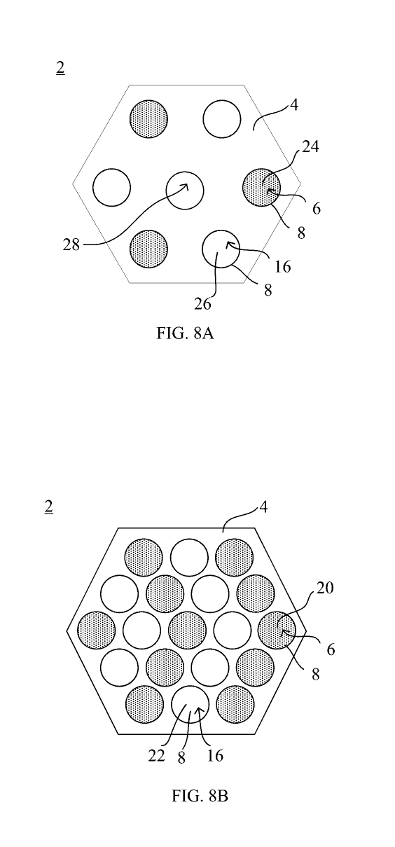

In an embodiment, as shown in FIG. 8A (biological sampling platform 2 that includes 7 through holes) and FIG. 8B (biological sampling platform 2 that includes 19 through holes), biological sampling platform 2 includes substrate 4, first through hole 6 disposed in substrate 4, and first sample 24 disposed in first through hole 6. Here, first sample 24 can be provided to a biological system in response to biological sampling platform 2 being disposed in the biological system. Biological sampling platform 2 further can include fiducial mark 10 (not shown) to index biological sampling platform 2. Substrate 4 is cleavable to provide a discrete layer having a thickness effective for analysis of the discreet layer by transmission microscopy. In a certain embodiment, biological sampling platform 2 also includes second through hole 16 disposed in substrate 4 and second sample 26 disposed in second through hole 16, wherein second sample 26 is provided to the biological system in response to biological sampling platform 2 being disposed in the biological system. Substrate 4 can include through hole 28 in which a sample is absent.

In biological sampling platform 2, substrate 4 can include a material that is selected to be compatible with biological tissue and stable in an in vivo or in vitro environment. According to an embodiment, substrate 4 is selected to be a material used in an FDA-approved medical device. Exemplary material includes a metal, polymer, glass, ceramic and the like. In a particular embodiment, substrate 4 includes a polymer, specifically a thermoset polymer or a thermoplastic polymer, more specifically a fluorinated thermoplastic polymer, yet more specifically a perfluorinated thermoplastic polymer, and further more specifically polytetrafluoroethylene (commercially available under the trademark TEFLON). In some embodiments, substrate 4 includes a medical-grade elastomer so that substrate 4 is flexible, resilient to deterioration, and biocompatible. It is contemplated that such material can include silicone or other relatively inert or biocompatible materials or an elastomer such as a polyisobutylene-based thermoplastic elastomer, poly(ethylene terephthalate) (PET), poly(tetrafluoroethylene) (PTFE), polypropylene (PP), polyurethane (PU), or a combination comprising at least one of the foregoing. Further, the elastomer can be a thermoplastic elastomeric biomaterial, e.g., polystyrene-b-polyisobutlyene-b-polystyrene (SIBS).

According to an embodiment, substrate 4 includes a material such that substrate 4 is cleavable to provide the discrete layer having a thickness effective for analysis of the discreet layer by transmission microscopy; and substrate 4 is selected to be resistant to histological processing to maintain a relative position of samples, e.g., during fixing, embedding, de-paraffinization, sectioning, or staining so that the samples in through holes (6, 16) are subjected to similar treatment. It is contemplated that biological sampling platform 4 that contains the biological sample disposed in a through hole (e.g., through hole 6, 16, 18, and the like) from the biological system can be subjected to cleaving to obtain the discrete layers. Cleaving biological sampling platform 2 can include cutting, slicing, or peeling substrate 4 to include a thin section the biological sample, first sample, second sample, or combination thereof. Such cleaving can include, e.g., microtome cleaving, laser cleaving, and the like.

In an embodiment, biological sampling platform 2 includes first sample 24. In a certain embodiment, biological sampling platform numerals includes a plurality of samples, e.g., first sample 24, second sample 26, and the like, which can be substantially identical or different with respect to size, shape, composition, or a combination thereof. According to an embodiment, first sample 24 and second sample 26 independently include an analyte to be absorbed by the biological system. In a particular embodiment, first sample 24 and second sample 26 independently include a matrix to receive the biological sample from the biological system. In some embodiments, first sample 24 and second sample 26 independently include the analyte to be absorbed by the biological system and the matrix to receive the biological sample from the biological system.

Exemplary analytes include electrolytes, bioactive small molecules, growth factors, synthetic organic molecules, cells, primary human bone marrow stromal cells, induced-pluripotent stem cells, stem cells, somatic cells, adult stem cells, osteoprogenitor cells, and the like.

Exemplary matrices include hydroxyapatite (HA), tricaclium phosphate (TCP), HA/TCP particles, porous scaffolds composed of collagen or other natural materials, porous scaffolds, hydrogels, fibrin gels, matrigel, alginate, crosslinked polymer gels, cross-linked poly(ethylene glycol) hydrogels, ceramic scaffolds, nanofiber scaffolds, fibrous scaffolds, microfiber scaffolds, electrospun fibrous materials, decellularized extracellular matrices, scaffolds comprised of natural materials of human- or animal-derived materials, and the like.

In a particular embodiment of biological sampling platform 2, first sample 24 includes stem or progenitor cells and a first growth factor disposed in first through hole 6, and second sample 26 includes stem or progenitor cells and a second growth factor disposed in a second through hole 16. Here, the logical sampling platform 2 is configured to be implanted subcutaneously into an organism, e.g., a mouse, to screen samples (e.g., first sample 24, second sample 26, and the like) for in vivo inducement of osteogenesis or bone formation.

The biological system receives, e.g., by implantation, biological sampling platform 2. Exemplary biological systems include tissue, organisms such as rodents (e.g., mice, rats, and the like), mammals (e.g., humans, dogs, cats, monkeys, and the like), and the like.

The biological sample can be provided to the through hole (e.g., first through hole from 6 or second through hole 16) from the biological system. Exemplary biological samples include cells (e.g., osteogenic cells, blood cells), tissue (e.g., muscle, bone, cardiac tissue, and the like), and the like.

Biological sampling platform 2 can be made in various ways. According to an embodiment, disposing a through hole in a substrate. In some embodiments, a process for making biological sampling platform 2 includes disposing first through hole 6 in substrate 4 and disposing first sample 24 in first through hole 6, wherein substrate 4 is cleavable to provide a discrete layer having a thickness effective for analysis of the discreet layer by transmission microscopy. The process further includes disposing second through hole 16 in substrate 4 and disposing second sample 26 in second through hole 16.

The sample (e.g., first sample 24, second sample 26, and the like) can be made by combining the analyte and matrix or combining the analyte with a suitable carrier, which can be, e.g., inert, nutrient, bacteriostatic, and the like for retention in the through hole or provision to the biological system. Such sample can be a solid, gel, or liquid (having a selected viscosity to be retained in the through hole) and can include particles, fibers, and the like. The analyte can be particulate and can be ground or milled to provide suitable particle size. In some embodiments, the sample includes an analyte encapsulated or disposed with a time-release material, e.g., an excipient such as copolyvidone, glyceryl monostearate, hydroxy propyl cellulose, and the like.

Biological sampling platform 2 can include a plurality of through holes, e.g., 2 through holes, 3 through holes, 7 through holes, 19 through holes, 47 through holes, 53 through holes, 200 through holes or more, without limitation. The through holes can be arranged in substrate 4 in a geometrical pattern that is symmetric or asymmetric. A size (e.g., a largest linear dimension such as a diameter) of the through hole is effective to receive or retain the sample (e.g., first sample 24) and biological sample, e.g., vascularized tissue. The size can be from 1 mm to 500 mm, specifically from 20 mm to 500 mm. A volume of the through hole can be effective to retain the sample (e.g., first sample or second sample) or biological sample, e.g., a volume from 5 microliters (μL) to 500 μL, specifically from 10 μL to 50 μL.

Substrate 4 can have a size effective to include a selected number of through holes having a selected size to provide for implantation of biological sampling platform 2 in the biological system and to receive the biological sample and are not particularly limited. It is contemplated that the biological sample may grow in the through hole when biological sampling platform 2 is present in the biological system. An exemplary size of substrate 4 can be, in a greatest linear dimension, from 300 μm to 4 cm. A least linear dimension of substrate 4 can be from 10 μm to 2 cm. A spacing between adjacent through holes can be selected to any amount, e.g., from 50 μm to 5 cm, specifically 200 μm to 1 cm, and more specifically 500 μm to 10 mm.

According to an embodiment, biological sampling platform 2 is disposed in a mouse. Here, biological sampling platform 2 (BSP) can have exemplary dimensions listed in the Table.

TABLE

| | | | BSP WITH |
|---|---|---|---|
| | | BSP WITH 7 | 19 |
| DIMENSION | EXEMPLARY | THROUGH | THROUGH |
| (WITH UNITS) | VALUES | HOLES | HOLES |
| Distance (cm) between | From 0.05 to 4 | 0.05 | 0.05 |
| walls of adjacent through | | | |
| holes | | | |
| Center-to-Center distance | From 0.25 to 4 | 0.48 | 0.29 |
| (cm) between adjacent | | | |
| through holes | | | |
| Volume (μL) of through | From 4 to 8000 | 43.5 uL | 13.5 uL |
| hole | | | |
| Diameter (cm) of through | From 0.25 to 4 | 0.43 | 0.24 |
| hole | | | |
| Length (cm) of greatest | From 0.25 to 4 | 1.5 | 1.5 |
| linear dimension of BSP | | | |
| Width (cm) of smallest | From 0.25 to 2 | 1.3 | 1.3 |
| linear dimension of BSP | | | |
| Thickness (cm) of BSP | From 0.1 to 1 | 0.3 | 0.3 |

First through hole 6 and second through hole 16 independently include a shape effective respectively to retain first sample 24 and second sample 26. Wall 8 can be straight so that first through hole 6 and second through hole 16 are open cylindrical structures. In some embodiments, wall 8 is curved with respect to a length axis of the through hole. Wall 8 can be concave, parabolic, convex, undulating, smooth, barbed, and the like.

A thickness of biological sampling platform 2 can be a thickness to support a through hole to retain a sample or receive a biological system, e.g., a thickness from 50 μm to 1 cm, specifically 1 mm to 5 mm, and more specifically 1 mm to 3 mm. The discreet layer produced by cleaving substrate 4 can have thickness effective for microscopy, e.g., a thickness such as 1 μm to 100 μm, specifically 1 μm to 20 μm.

The biological sampling platform is resistant to cleaning (e.g., with alcohol, acetone, acids, and the like) or sterilization (e.g., being subjected to high temperatures such as in an autoclave).

Biological sampling platform 2 has numerous uses included, in an embodiment, a process for collecting a biological sample that includes disposing first through hole 6 in substrate 4, disposing first sample 24 in first through hole 6 to form biological sampling platform 2, disposing biological sampling platform 2 in a biological system, providing first sample 24 to the biological system in response to biological sampling platform 2 being disposed in the biological system, and receiving a first biological sample from the biological system in first through hole 6 to collect the first biological sample, wherein substrate 4 is cleavable to provide a discrete layer having a thickness effective for analysis of the discreet layer by transmission microscopy. The process also includes disposing second through hole 16 in substrate 4 and disposing second sample 26 in second through hole 16 prior to disposing biological sampling platform 2 in the biological system, receiving a second biological sample from the biological system in second through hole 16. Thereafter, the process includes removing biological sampling platform 2 from the biological system. Additionally, the process includes cleaving substrate 4 to include the first biological sample, the second biological sample, or combination of the foregoing biological samples to form the discreet layer and performing transmission microscopy on the discreet layer. As used herein, transmission microscopy can be substituted for other types of microscopy including dark-field microscopy, phase contrast microscopy, scanning tunneling electron microscopy, electron microscopy, transmission electron microscopy, X-ray microcomputed tomography, and the like, where a thickness of the discreet layer has a thickness effective to perform the type of microscopy.

Biological sampling platform 2 can be made by molding or machining substrate 4 to a selected shape and size, using laser cutters, drill bits, lathes, and the like. Through holes can be disposed in substrate 4 during the molding or machining to produce biological sampling platform two having through holes disposed in substrate 4. Thereafter, samples can be prepared and disposed in the through holes by pouring, packing, and the like the sample in the through holes.

In a particular embodiment, the biological system is a mouse that is subjected to implanting biological sampling platform 2 subcutaneously in a space between dermal layers and muscle. A control sample can be included as a mast implanted in a separate location in the space. In some embodiments, first sample 24, second sample 26, or combination thereof can be a positive control or a negative control a contemplation of screening tests performed with biological samples derived from the biological system in the through holes (6, 16). After a sample time elapses, biological sampling platform 2 is removed from the biological system and subjected to processing and analyzing.

In some embodiments, processing includes imaging (X-ray microcomputed tomography, X-ray) or fixing the biological sample disposed in the through holes, e.g., by contacting the biological sample in the through holes with a fixative (e.g., paraformaldehyde (PFA) in phosphate-buffered saline (PBS) (e.g., including sodium chloride, sodium hydrogen phosphate, potassium chloride, potassium dihydrogen phosphate, and the like) to fix the tissue; demineralized in the biological sample, e.g., by contacting the biological sample with a demineralized in composition such as ethylenediaminetetraacetic acid (EDTA) in PBS to leach or dissolve minerals from the biological sample; dehydrating the biological sample, e.g., by contacting the biological sample with a dehydrant such as an alcohol; disposing the biological sampling platform 2 in an encapsulant (e.g., paraffin) to support biological sampling platform 2 for cleavage; and cleaving biological sampling platform 2, e.g., by subjecting a logical sampling platform 2 to slicing using a microtome produce the discreet layer. The discreet layer can then be subjected to microscopy and procedures for preparing the discreet layer for microscopy such as staining

In some embodiments, the samples disposed in the biological sampling platform are subjected to biochemical or imaging analysis, and the biological sampling platform provides a tissue barrier to provide biological samples of uniform size and volume. Such uniform samples provide generation of comparative data in biochemical analyses (e.g., polymerase chain reaction, enzymatic assays, microarray gene expression analysis, protein analysis via mass spectrometry, protein expression analysis via enzyme-linked immunosorbent assay, and the like).

Biological sampling platform 2 has beneficial and advantageous properties., including providing through holes for sampling a small volume (e.g., a microliter volume) and high throughput screening of a plurality of biological samples or other samples (e.g., first sample) per biological sampling profile 2. Moreover, a single biological system (e.g., an organism such as an animal host) can be used to provide a plurality of biological samples or to receive a plurality of samples (e.g., first sample or second sample). Accordingly, biological sampling platform 2 allows a decrease in a total number of biological systems used per study. Due to collection of the plurality of biological samples per biological sampling platform 2, reduction of system-to-system variability for systematic in vivo screening is accomplished.

Biological sampling platform 2 can be used advantageously for complexities such as high-throughput in vivo screening of osteogenesis or growth of other tissue as well as in vivo or in vitro studies involving effects of the first sample on tissue. Furthermore, through holes provided in biological sampling platform 2 are configured to receive tissue-ingrowth and to support vascularization of the tissue.

Using biological sampling platform to obtain biological samples from biological systems provides low variability among the biological samples by allowing a plurality of samples to be tested in the same biological system (e.g., a same mouse). Additionally, fewer animals are used, which reduces regulatory, space, or maintenance costs and decreases analysis times for comparison of samples (e.g., first sample) and biological samples, e.g., in histology studies. Given the selected volume of through holes in biological sampling platform 2, through holes can have a selected volume to retain a low volume of material such that small amounts of material such as a number of cells are collected. Alternatively, a large volume can be used for collection of a larger tissue size. Beneficially, within biological sampling platform 2, cross-migration of samples or biological samples does not occur among the through holes.

The biological sampling platform herein provides systematic treatment of samples for analyses. In an embodiment, for histological analysis, the biological sampling platform is fixed, embedded in paraffin, sectioned via microtome, stained (e.g., stained with hematoxylin and eosin, immunostaining, and the like), and imaged (e.g., by light microscopy, fluorescence microscopy, polarized light microscopy, and the like). In this manner, samples disposed in the biological sampling platform are treated uniformly, yielding comparative data. Further, the biological sampling platform is stable during the histological preparation (e.g., the biological sampling platform maintains sample positions during paraffin embedding, is resistant to xylenes used for de-paraffinization, cleavable by microtome, and the like).

The biological sampling platform herein also provides samples with controlled shape and volume (e.g. cylinders in 6 and 16). This is advantageous for biochemical analyses (e.g., polymerase chain reaction, enzymatic assays, microarray gene expression analysis, protein analysis via mass spectrometry, protein expression analysis via enzyme-linked immunosorbent assay, etc.), where an amount of tissue analyzed affects results. The biological sampling platform further provides a barrier such as a border of the sample, which is advantageous for imaging analysis (e.g., X-ray microcomputed tomography, histology, electron microscopy, and the like) where standardizing the volume or area that is analyzed improves comparability of results.

The articles and processes herein are illustrated further by the following Examples, which are non-limiting.

### EXAMPLES

### Example 1

### Preparation of Biological Sampling Platform

Biological sampling platforms were cut from a polytetrafluoroethylene (PTFE, Teflon) sheet of thickness 0.3 cm using a using a laser cutter to provide a selected shape. Shapes of the biological sampling platform include hexagons (FIGS. 5A and 5B) with 0.43 cm diameter through holes for a 7-hole embodiment (FIG. 5A, having 43.5 μL/through hole) and 0.24 cm diameter through hole (FIG. 5B, 13.5 μL/through hole) for a 19-hole embodiment. Through holes were open on a top surface and bottom surface to provide communication (e.g., diffusion) of nutrient or waste or vascularization. A center-to-center distance between adjacent through holes was 0.48 cm and 0.29 cm respectively for the 7-hole and 19-hole embodiments. A fiduciary mark was cut on the biological sampling platforms to indicate a relative position of samples in the through holes. Corners and edges of the biological sampling platforms were smoothed with 1200 grit silicon carbide sand paper to prevent tissue irritation to a host organism after implantation. After fabrication, the biological sampling platforms were cleaned sequentially with acetone (5 min), ethanol (10 min), or deionized water (10 min) in an ultrasonic water bath. The biological sampling platforms were autoclaved and stored under sterile conditions until use.

### Example 2

### Collection of Biological Sample

A gelatin sponge (Sterile Gelfoam available from Pfizer) was cut into blocks (4 mm×4 mm×4 mm) for disposal in through holes of a biological sampling platform that included 19 through holes. After cutting, the blocks were soaked in cell culture medium, squeezed with sterile filter paper using forceps to remove air bubbles, and disposed individually in separate through holes of the biological sampling platform. Recombinant human bone morphogenetic protein-2 (rhBMP-2, eBioscience, reconstituted in sterile water at 0.5 mg/mL) was pipetted (8 μL, 4 μg rhBMP-2) onto the blocks disposed in the through holes. Negative control blocks were provided with cell culture medium (8 uL) instead of rhBMP-2. Two month-old wild type FVB/N-Tg(LRRK2)lCjli/J mice (provided from The Jackson Laboratory, Maine, United States) were used as host in which to implant the biological sampling platform. The mice were subjected to an operation in which the biological sampling platform was inserted through a lateral incision proximate to a pelvis such that the biological sampling platform was present in a subcutaneous space deep to dorsal skin, and the incision was closed with a staple. After 8 weeks (in some mice 16 weeks), the biological sampling platforms were removed from the host mouse and subjected to analysis. FIG. 9 shows an X-ray image of the biological sampling platform disposed in the mouse.

### Example 3

### Sample Analyses: Polymerase Chain Reaction, X-ray Microcomputed Tmography, and Histological Processing.

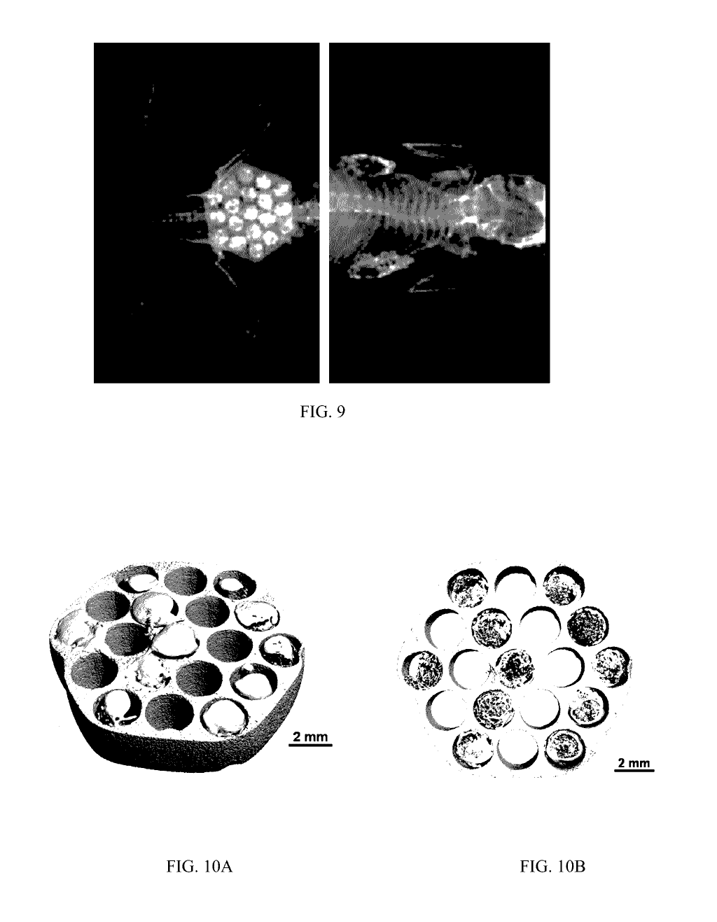

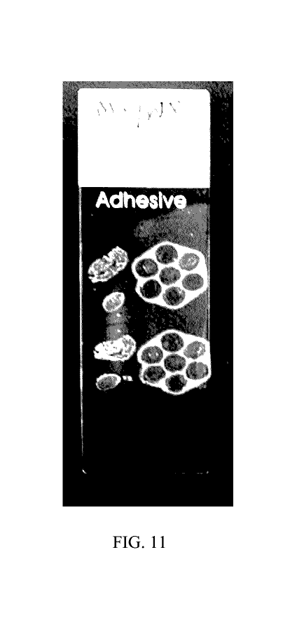

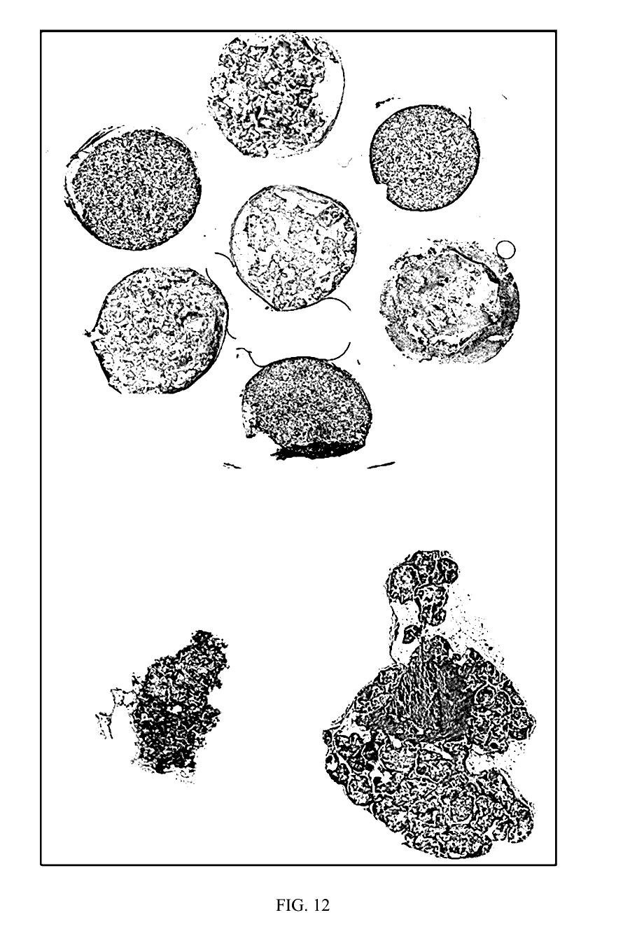

The biological sampling platform that was removed from the mice after 8 weeks of being disposed in the mouse were subjected to analysis that included removal the gelatin foam from the through holes in the biological sampling platform. The gelatin foam was frozen at −80° C. for subsequent analysis by quantitative reverse transcription polymerase chain reaction (qPCR). Frozen portions of the gelatin foam were ground into powder at liquid nitrogen temperature and mixed with a chemical solution to extract RNS (commonly referred to as guanidinium thiocyanate-phenol-chloroform extraction; Trizol reagent commercially available from Life Technologies). RNA was extracted and analyzed by PCR techniques. In some cases, samples were fixed with formaldehyde while present in the biological sampling platform and subjected to imaging by X-ray microcomputed tomography to image bone formation as shown in FIG. 10. For histological analysis, fixed samples in the biological sampling platform were demineralized with EDTA for several weeks, washed with phosphate buffered saline, dehydrated with ethanol, immersed in xylene, embedded in paraffin, sectioned (to provide 6 μm thick discreet layers) using a microtome, mounted on a glass slide and stained with hematoxylin and eosin. The biological sampling platform was compatible with the histological processing using an automated tissue processor and maintained the positions of samples throughout the process as shown in FIG. 11. Stained slides of the entire biological sampling platform were imaged by transmitted light microscopy as shown in FIG. 12.

While one or more embodiments have been shown and described, modifications and substitutions may be made thereto without departing from the spirit and scope of the invention. Accordingly, it is to be understood that the present invention has been described by way of illustrations and not limitation. Embodiments herein can be used independently or can be combined.

Reference throughout this specification to “one embodiment,” “particular embodiment,” “certain embodiment,” “an embodiment,” or the like means that a particular feature, structure, or characteristic described in connection with the embodiment is included in at least one embodiment. Thus, appearances of these phrases (e.g., “in one embodiment” or “in an embodiment”) throughout this specification are not necessarily all referring to the same embodiment, but may. Furthermore, particular features, structures, or characteristics may be combined in any suitable manner, as would be apparent to one of ordinary skill in the art from this disclosure, in one or more embodiments.

All ranges disclosed herein are inclusive of the endpoints, and the endpoints are independently combinable with each other. The ranges are continuous and thus contain every value and subset thereof in the range. Unless otherwise stated or contextually inapplicable, all percentages, when expressing a quantity, are weight percentages. The suffix “(s)” as used herein is intended to include both the singular and the plural of the term that it modifies, thereby including at least one of that term (e.g., the colorant(s) includes at least one colorants). “Optional” or “optionally” means that the subsequently described event or circumstance can or cannot occur, and that the description includes instances where the event occurs and instances where it does not. As used herein, “combination” is inclusive of blends, mixtures, alloys, reaction products, and the like.

As used herein, “a combination thereof” refers to a combination comprising at least one of the named constituents, components, compounds, or elements, optionally together with one or more of the same class of constituents, components, compounds, or elements.

All references are incorporated herein by reference.

The use of the terms “a” and “an” and “the” and similar referents in the context of describing the invention (especially in the context of the following claims) are to be construed to cover both the singular and the plural, unless otherwise indicated herein or clearly contradicted by context. “Or” means “and/or.” Further, the conjunction “or” is used to link objects of a list or alternatives and is not disjunctive; rather the elements can be used separately or can be combined together under appropriate circumstances. It should further be noted that the terms “first,” “second,” “primary,” “secondary,” and the like herein do not denote any order, quantity, or importance, but rather are used to distinguish one element from another. The modifier “about” used in connection with a quantity is inclusive of the stated value and has the meaning dictated by the context (e.g., it includes the degree of error associated with measurement of the particular quantity).

What is claimed is:

1. A biological sampling platform comprising:
a substrate; and
a first through hole disposed in the substrate to receive a first sample and to provide the first sample to a biological system in response to the biological sampling platform being disposed in the biological system, the substrate being cleavable to provide a discrete layer having a thickness effective for analysis of the discreet layer by transmission microscopy.

2. The biological sampling platform of claim 1, further comprising a second through hole disposed in the substrate to receive a second sample and to provide the second sample to the biological system in response to the biological sampling platform being disposed in the biological system.

3. The biological sampling platform of claim 2, wherein the first sample or the second sample independently comprises an analyte to be absorbed by the biological system.

4. The biological sampling platform of claim 2, wherein the first sample or the second sample independently comprises a matrix to receive a biological sample from the biological system.

5. The biological sampling platform of claim 2, wherein the first sample or the second sample independently comprises:
an analyte to be absorbed by the biological system; and
the matrix to receive a biological sample from the biological system.

6. The biological sampling platform of claim 2, wherein the first through hole or the second through hole independently comprise a shape effective to respectively retain the first sample and the second sample.

7. The biological sampling platform of claim 6, wherein a size of the first through hole and a size of the second through hole are large enough to receive vascularized tissue.

8. The biological sampling platform of claim 2, wherein a size of the first through hole and a size of the second through hole are large enough to respectively retain the first sample and the second sample.

9. The biological sampling platform of claim 2, wherein the first sample is different than the second sample.

10. A biological sampling platform comprising:
a substrate;
a first through hole disposed in the substrate; and
a first sample disposed in the first through hole, the first sample being provided to a biological system in response to the biological sampling platform being disposed in the biological system.

11. The biological sampling platform of claim 10, further comprising a fiducial mark to index the biological sampling platform,
wherein the substrate is cleavable to provide a discrete layer having a thickness effective for analysis of the discreet layer by transmission microscopy.

12. The biological sampling platform of claim 11, further comprising:
a second through hole disposed in the substrate; and
a second sample disposed in the second through hole, the second sample being provided to the biological system in response to the biological sampling platform being disposed in the biological system.

13. The biological sampling platform of claim 12, wherein the first sample and the second sample independently comprise an analyte to be absorbed by the biological system.

14. The biological sampling platform of claim 12, wherein the first sample and the second sample independently comprise a matrix to receive a biological sample from the biological system.

15. The biological system of claim 12, wherein the first sample and the second sample independently comprise:
an analyte to be absorbed by the biological system; and
the matrix to receive a biological sample from the biological system.

16. The biological system of claim 12, wherein the first through hole and the second through hole independently comprise a shape and size effective for the first through hole and the second through hole respectively to retain the first sample and the second sample.

17. The article system of claim 16, wherein a size of the first through hole and a size of the second through hole are large enough to receive vascularized tissue.

18. The article system of claim 16, wherein the substrate is configured to maintain a relative position and size of the first sample and the second sample.

19. A process for making a biological sampling platform, the process comprising:
disposing a first through hole in a substrate; and
disposing a first sample in the first through hole, the substrate being cleavable to provide a discrete layer having a thickness effective for analysis of the discreet layer by transmission microscopy, the substrate being resistant to processing of the discreet layer for a histological study and configured to maintain a relative position and size of the first sample in the first through hole.

20. The process of claim 19, further comprising:
disposing a second through hole in the substrate; and
disposing a second sample in the second through hole.

21. A process for collecting a biological sample, the process comprising:
disposing a first through hole in a substrate;
disposing a first sample in the first through hole to form a biological sampling platform;
disposing the biological sampling platform in a biological system; and
receiving a first biological sample from the biological system in the first through hole to collect the first biological sample, the substrate being cleavable to provide a discrete layer having a thickness effective for analysis of the discreet layer by transmission microscopy, the substrate being resistant to processing of the discreet layer for a histological study and configured to maintain a relative position and size of the first sample in the first through hole.

22. The process of claim 21, further comprising:
disposing a second through hole in the substrate;
disposing a second sample in the second through hole prior to disposing the biological sampling platform in the biological system; and
receiving a second biological sample from the biological system in the second through hole.

23. The process of claim 22, further comprising removing the biological sampling platform from the biological system.

24. The process of claim 23, further comprising cleaving the substrate to include the first biological sample, the second biological sample, or combination comprising at least one of foregoing biological samples to form the discreet layer.

25. The process of claim 24, further comprising performing transmission microscopy on the discreet layer.
